(19) United States
(12) Patent Application Publication
(10) Pub. No.: US 2003/0153061 A1
(43) Pub. Date: Aug. 14, 2003

(54) Method for immobilizing recognition components

(76) Inventor: Wolfgang Schuhmann, Bochum (DE)

Correspondence Address: SUGHRUE MION, PLLC, 2100 PENNSYLVANIA AVENUE, N.W., WASHINGTON, DC, 20037, US

(21) Appl. No.: 10/275,698

(22) PCT Filed: May 8, 2001
(86) PCT No.: PCT/CH01/00279

(30) Foreign Application Priority Data
May 10, 2000 (DE) 100 22 750.3

(51) Int. Cl. C12N 9/04; C12N 9/08

(52) U.S. Cl. 435/190; 435/192

(57) ABSTRACT

The invention relates to a method of producing a surface (1) functionalised with detection elements, comprising an optionally electrically conductive body (2) and a resin layer (3) applied thereto, including at least one substance (4) with a detection function, characterized in that

a) a resin layer (3) is deposited on the body (2) by electro-deposition,

b) the resin layer (3) comprises resins derived from polymerisable, ion-carrying monomers which are deposited by changing the pH value and

c) the substance(s) and optionally further additives is/are deposited with the resin from a resin emulsion on the body (2) during the electro-deposition.

This invention relates to a novel, simple method of immobilising molecular functional elements, especially biological detection elements on optionally conductive surfaces, and to the articles obtainable with this method.

The immobilisation of detection elements, especially biological and biochemical detection elements on optionally conducting surfaces allows a number of different applications in the field of sensor technology, especially biosensor technology, chemical or biochemical combinatorial technology and medical diagnostics, for example for screening. The immobilisation of the detection elements can be effected for example by absorption, as is disclosed in U.S. Pat. No. 3,839,175, cross-linking, D. J. Strike, N. F. de Rooij, M. Koudelka-Hep, Biosens. Bioelectron, 10 (1995) 61-66 or by inclusion in a polymer film, as in W. Schumann, Mikrochim. Acta, 121 (1995) 1-29. In the matrix used for the immobilisation of the detection elements it is mostly a polymer which is used, which is deposited by radical polymerisation on the optionally conducting surface. The radical reaction can be induced electrochemically, chemically or photochemically, cf. EP-A 0 691 408 for example. A disadvantage of these known radical polymerisations is the action of oxygen as an inhibitor of such a reaction. On account of this action the whole reaction has to take place under a protective gas atmosphere. Further methods for immobilisation are lithographic methods on solid bodies, such as are disclosed in G. H. McGall, A. D. Barone, M. Diggelmann, S. P. A. Fodor, E. Gentalen, N. Ngo, J. Am. Chem. Soc., 119 (1997) 5081, and the printing method as disclosed by G. F. Khan, Electroanalysis, 9 (1997) 325-329. Both methods require a high usage of materials and only partially suited to local deposition on optionally conducting surfaces with a high resolution (of<100 μm).

The invention is based on the object of providing coatings of sensors in the form of a functionalised, especially enzymatically active electrode surface, wherein the following objectives inter alia are to be achieved: rapid, simple and cost-effective manufacture; immobilisation on the most varied, optionally conductive surfaces, e.g. customary electrode materials; exclusion of oxygen unnecessary during the film production; the film thickness can be varied without constraint; modified or unmodified components, for example small latex balls, small glass balls, graphite particles, etc. can be encased in the layer in a simple manner; it is to be possible to form multilayer films with different functions; local depositions with spatially limited pH gradients are possible and thus the formation of array structures, e.g. for combinatorial chemistry, biosensor arrays, immuno-assays or various screening methods; possibility of surface modification; possibility of spatial resolution of the film; as well as the possibility of the use of fluorescent markers or other fluorescing detection components. This object is met by the subject matter of the present claims.

The method of electro-deposition lacquering (EPD), F. Beck, Electrochim. Acta, 33 (1988) 839-850, is used industrially for protection against corrosion in automobile chassis and components, radiators and drink cans. In anodic electro-deposition the protons released by decomposition of water at the anode are used to neutralise and thus precipitate a soluble polymer, negatively charged on account of carboxylate groups. The processes underlying this method can be summarised as follows:

| | |
|---|---|
| Decomposition of water: | 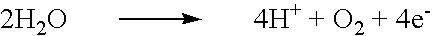 |
| Polymer deposition: | 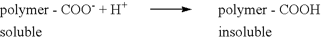 |

The film is then cross-linked on the surface to form an impermeable coating which has great mechanical and chemical resistance, usually by heating (as a rule 165-185EC). As well as the so-called anodic electro-deposition lacquer (AED) there is the cathodic electro-deposition lacquer (CED). This is deposited on the optionally conducting surface by locally generated hydroxide ions. This process is based on the following general reaction scheme:

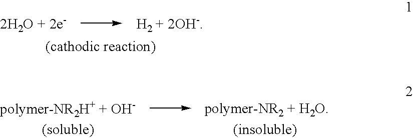

A further group of compounds are those of an amphoteric nature, e.g. peptides, oligopeptides or proteins. By suitable choice of the electrode reaction the substance provided with groups charged both positively and negatively in various proportions, e.g. the peptide, especially an oligopeptide or polypeptide, can be deposited on the electrode surface in its insoluble state (isoelectric point).

The result of the electrode reaction, namely a deposited, essentially organic film, can optionally be treated further, in which modifications can be effected—physically (e.g. heat) or chemically (e.g. chemical derivatization).

In the present invention therefore a substance with a detection function, e.g. an enzyme as well as resin particles in the form of a resin emulsion, is present as an electrolysis fluid and, on account of the creation of protons (hydronium ions) or hydroxyl ions at the surface of the target electrode, resin deposits are created, which incorporate and thus immobilise enzyme molecules for example simultaneously present in the electrolysis solution. On account of the fact that this process takes place electrochemically, electro-deposits of arbitrary form can be deposited in an arbitrary pattern for example on a target electrode of relative large area.

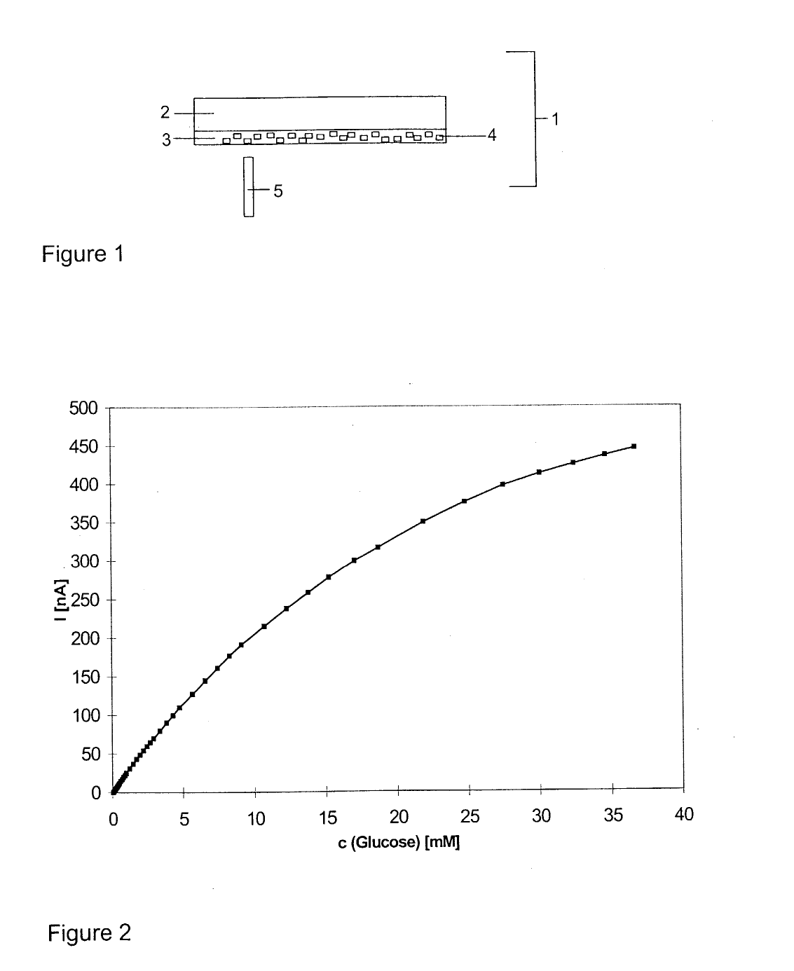

FIG. 1 explains the structure in principle of a functionalised surface 1 obtained by the method according to the invention, functionalised with detection elements. In FIG. 1 reference numeral 1 indicates the product obtained by the method, namely the surface functionalised with detection elements. Reference numeral 2 indicates the optionally electrically conductive body. Reference numeral 3 shows the deposited lacquer coating, which incorporates the substance 4 with a detection function. Probe tips are meant to indicate that this method can be effected in a very targeted way in a field between the probe 5 and the body 2.

FIG. 2 shows the recording of a calibration curve according to Example 1, namely the calibration curve of a platinum disc electrode (φ 1 mm) modified with GOD (glucose oxidase). The measurement took place in 20 ml phosphate buffer, pH 7, with the addition of 0.1 mM glucose solution. The potential applied to the coated electrode amounted to 600 mV against Ag/AgCl.

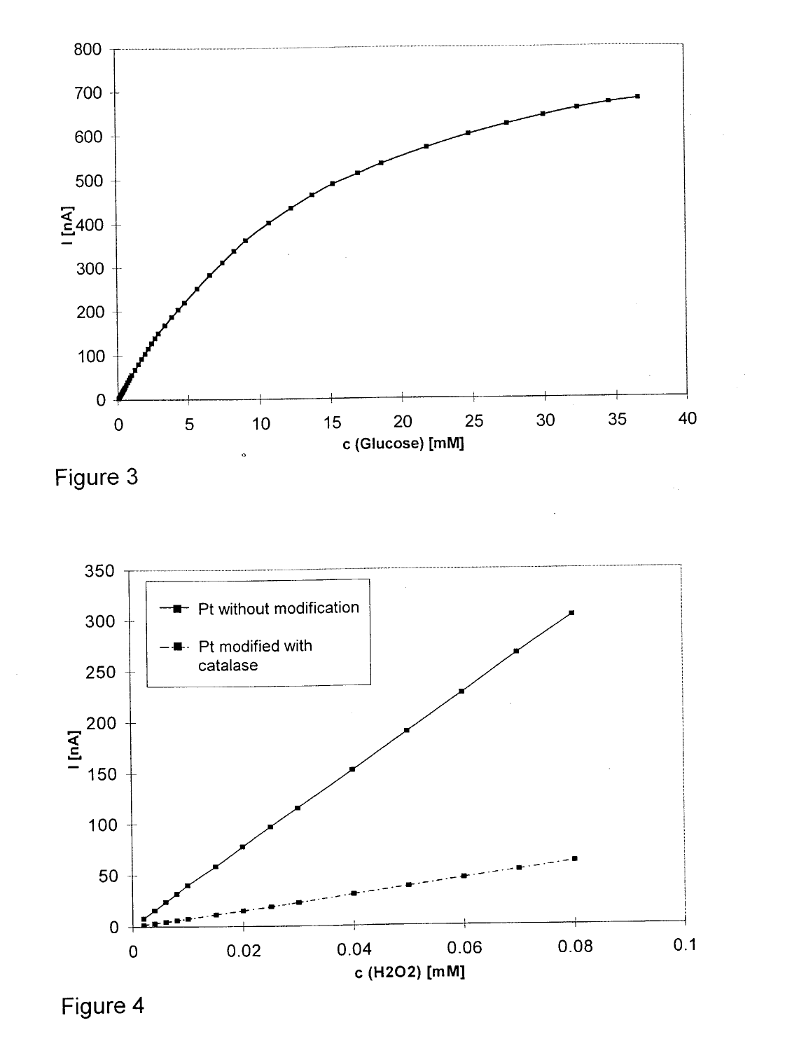

FIG. 3 shows the calibration curve of a carbon electrode modified with GOD, produced by means of a thick film technique (basic structure of the sensor is from the company SensLab). The measurement was effected in 20 ml phosphate buffer, pH 7, with the addition of 0.1 mM glucose solution. The potential applied to the coated electrode amounted to 600 mV against Ag/AgCl.

FIG. 4 shows the comparison of the hydrogen peroxide sensitivity between an electrode modified with catalase and an unmodified platinum disc electrode (φ 1 mm). The measurement was effected in 20 ml phosphate buffer, pH 7, with the addition of 2 mM hydrogen peroxide solution. The potential applied to the coated electrode amounted to 600 mV against Ag/AgCl.

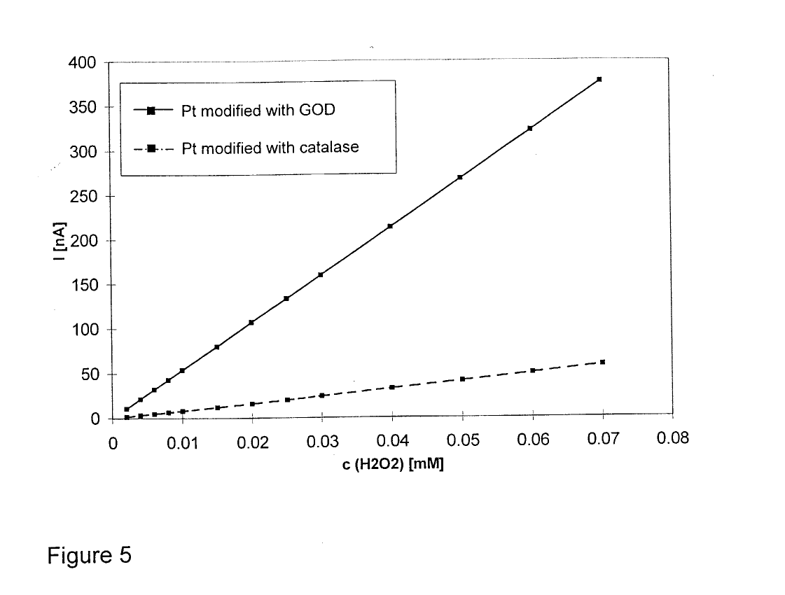

FIG. 5 shows a comparison of the hydrogen peroxide sensitivity between an electrode modified with catalase and a GOD modified platinum disc electrode (φ 1 mm). The measurement was effected in 20 ml phosphate buffer, pH 7, with the addition of 2 mM hydrogen peroxide solution. The potential applied to the electrodes amounted to 600 mV against Ag/AgCl.

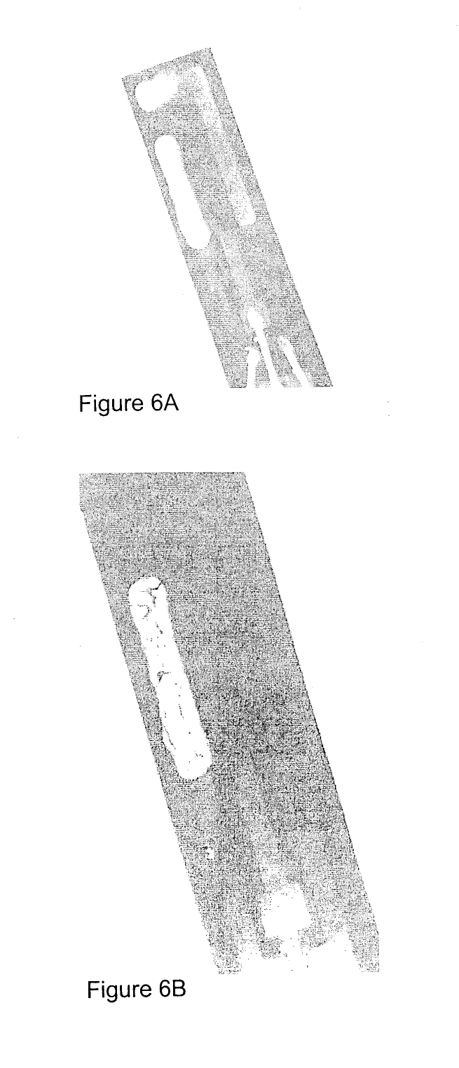

FIG. 6 shows a microscope photograph of a modified platinum micro-array. Two different films are deposited on this structure. The L-shaped strip electrode was coated with a GOD film while the smaller, straight strip electrode was provided with a bead film. The upper exposure shows the structure under normal light, the lower under fluorescent light.

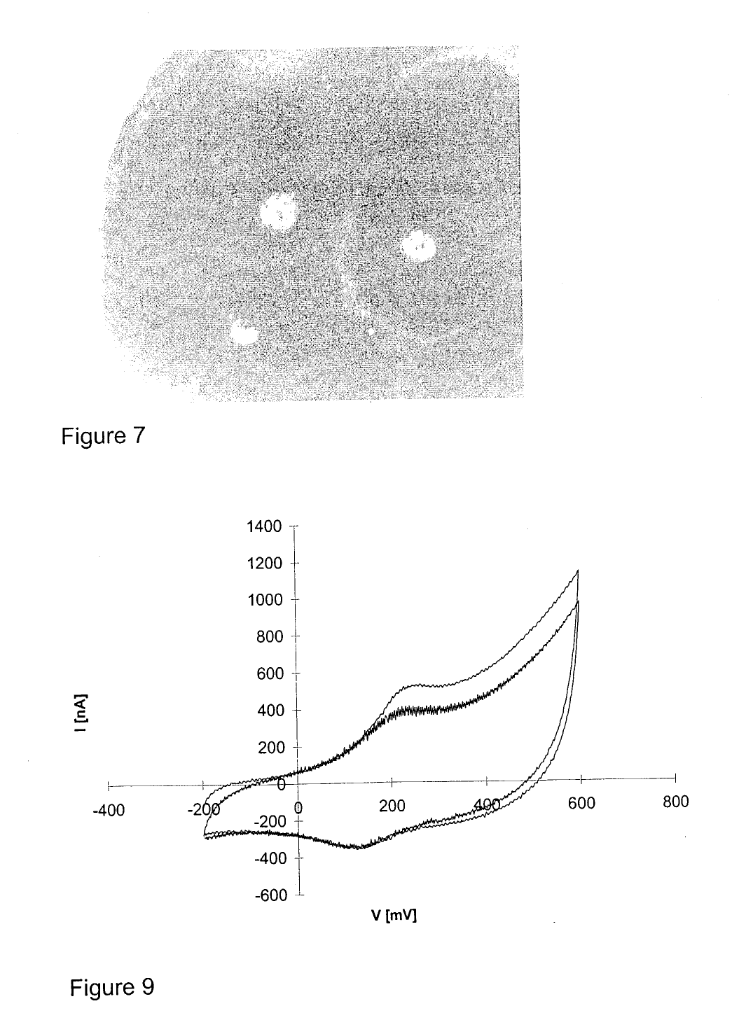

FIG. 7 shows a microscope photograph of three polymer points deposited locally by means of an SECM on a gold disc electrode (φ 3 mm).

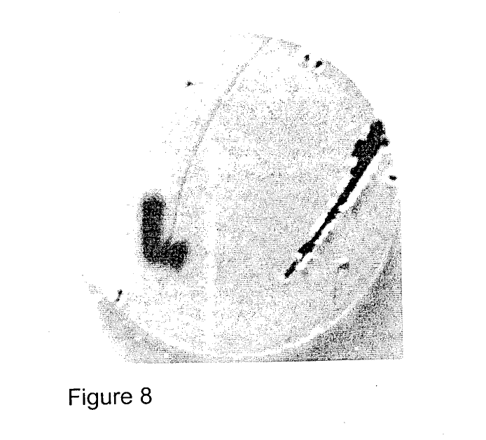

FIG. 8 shows a microscope exposure of an (L-shaped) polymer film locally dissociated by means of SECM. A potential pulse of −2000 mV against Ag/AgCl was applied for 1 s several times to the micro-electrode moved laterally over the surface for the dissolution. The dissolution was effected in 5 mM ruthenium hexaamine solution.

FIG. 9 shows a cyclo-voltamogram (CV) of a platinum disc electrode (φ 1 mm) modified with redox-active groups ([Os(bpy)₂(histamine)Cl]Cl. The measurement was effected from −200 to +600 mV against Ag/AgCl, in a 0.1 M lithium perchlorate solution.

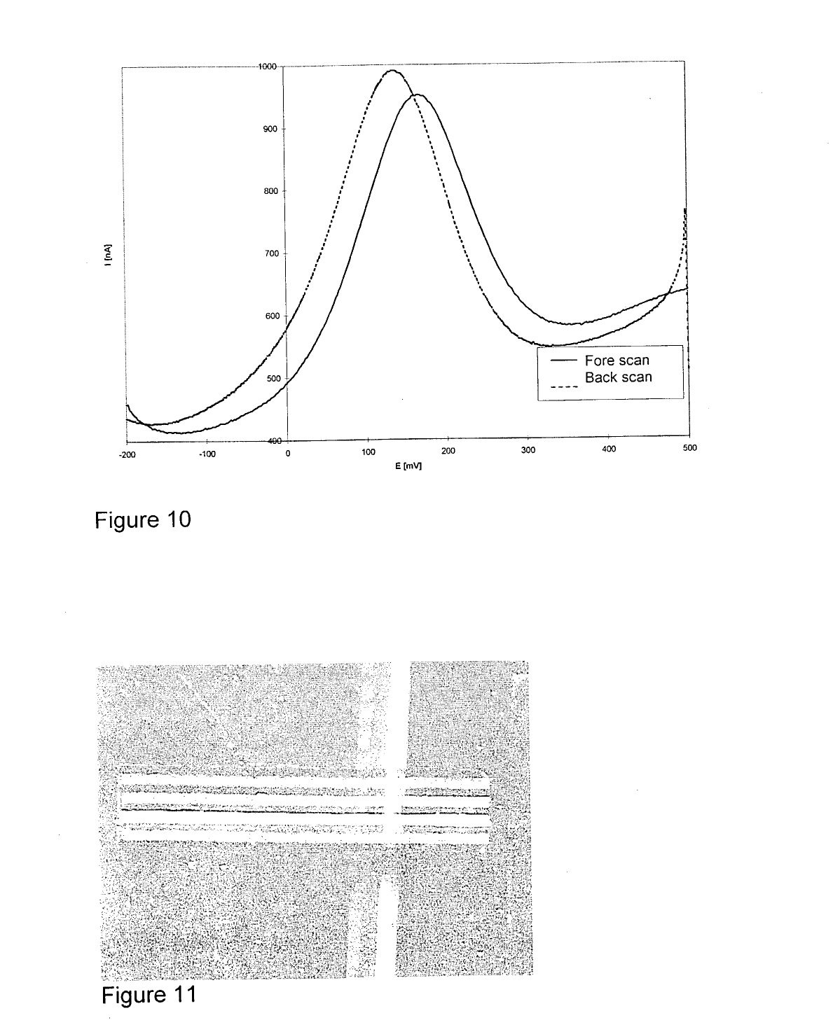

FIG. 10 shows a differential pulse voltamogram (DPV) of a platinum disk electrode (φ 1 mm) modified with a redox group ([Os(bpy)₂(histamin)Cl]Cl).

FIG. 11 shows an array arrangement, wherein the second strip has been coated.

FIG. 12 shows an example of targeted dissolution of a previously deposited film.

Arbitrary materials can be used as optionally electrically conductive bodies 2, which are capable of creating a potential (for generating H⁺ or OH⁻ for example) and a field (optionally for migration of the particles) at an electrode, for example the electrode 5, which can deposit the emulsion on the surface of 2. All noble element surfaces are especially suitable for this, above all surfaces of gold, silver, platinum, palladium, iridium, rhenium, mercury, ruthenium and osmium. Furthermore metals which behave inertly electrochemically to the maximum extent are suitable, such as chromium, nickel, cobalt, iron and alloys known as stainless steel, as well as titanium, zirconium and hafnium surfaces. Moreover certain passivated metals or alloys, e.g. aluminium, gallium or aluminium alloys which behave inertly during the electrochemical process are suitable. The metals should basically not inhibit the incorporated detection elements, e.g. enzymes. The size of a target electrode depends on the desired purpose. As a rule the surfaces amount to 0.5 to 50 mm². However, with suitable dimensioning, it is entirely possible to increase or decrease this range by 3, 5, 10, 20, 100, 250 or 500 times. An electrode surface is advantageously polished before use and additionally treated, for example by platinizing.

What matters is that the electrode surface is in such condition that the electrochemical and/or electrostatic operations required for the deposition can take place thereat.

It is also possible to use semiconductor electrodes, such as silicon surfaces, GaAs, ITO, In₂O₃ among others, which are doped if necessary with suitable trace elements, or carbon electrodes, with suitable dimensioning and surface treatment. It is also possible to use arbitrary surfaces which are not of a metallic nature, insofar as an electric potential can be applied to them which is sufficient to deposit the resin out of the emulsion on them.

Arbitrary known resin emulsions which are suitable for electro-deposition can be used as the resin emulsion 3. Among these are organic compounds of higher molecular weight in the range from 300 to 50000; 600 to 30000; 1000 to 20000; 800 to 10000; 1000 to 15000, also however higher molecular weights, insofar as they can be dissolved or emulsified, from the previously given values up to 50000, 70000, 100000, 250000 or even 1000000, especially resins with electric charges of an ionic nature, which are created by functional groups on the constituent monomers. Examples are modified polystyrols, olefins, polyamides and vinyl and acryl compounds, also however biochemically interesting polymers, such as proteins, polysaccharides with functional groups, vegetable gums, oligo and poly nucleotides. α, β olefinic unsaturated carboxylic acid polymers have proved themselves in particular for the anodic deposition.

A preferred resin for the resin emulsion used in the invention for the anodic electro-deposition is for example a resin preparation on the basis of, a copolymerisate containing carboxylic groups, masked isocyanate groups, hydroxyl and ether groups, which can be dissolved or dispersed in water by at least partial salt formation with ammonia or an organic base, wherein the copolymerisate contains polymerised:

1. At least one α, β olefinic unsaturated carboxylic acid with 3 to 5 carbon atoms or a half-ester of an α, β olefinic unsaturated dicarboxylic acid containing 3 to 5 carbon atoms,

2. 10 to 35 percent by weight of an N(1-alkenyl) isocyanate masked with CH-, OH- or NH-active masking means,

3. 20 to 50 percent by weight of an adduct of an epoxide resin on the basis of bisphenol A and epichlorhydrin with a molecular weight between 380 and 3500 and an olefinic, unsaturated alcohol containing 3 to 5 carbon atoms.

4. 5 to 64 percent by weight of one or more olefinic, unsaturated compounds not recited under 1. to 3. which can be copolymerised,

with the provision that the copolymerisate has a mean molecular weight between 1000 and 20000, includes the component (1) polymerised in such an amount that the acid number of the copolymerisate amounts to 35 to 150 mg KOH/g and the sum of the percentages recited under 1 to 4 is 100.

The copolymerisate mentioned above has a mean molecular weight between 1000 and 20000. The components are polymerised in such an amount that the acid number of the copolymerisate amounts to 35 to 150 mg KOH/g. The sum of the percentages recited under 1 to 4 amounts to 100. It is to be emphasised that the equivalent ratio of the reactive hydrogen atoms of the component (3) to the masked isocyanate groups in the copolymerisate preferably amounts to about 1:1. Furthermore an adduct of vinylisocyanate or propenylisocyanate and cyclohexanol, ter-butanol, triazabenzol or ε-caprolactam is preferably used with the molecular ratio 1:1 of isocyanate/masking means. In particular the copolymerisate contains as component (1) acrylic or methacrylic acid, as component (2) vinylisocyanate masked with ε-caprolactam, as component (3) a conversion product of an epoxide resin of bisphenol A and epichlorhydrin with a mean molecular weight of about 900 and allyl alcohol, as well as polymerised 2-ethylhexylacrylate or butylacrylate as component (4).

Also preferred is a resin-like mass which is used as a mixture of a water soluble resin-like material and a water insoluble resin-like mass. The mixture is dispersed in an aqueous medium, in which water is the main constituent. The water soluble resin-like materials are polymers and are rendered water soluble through incorporation of sufficient hydrophilic groups into the polymer. The hydrophilic groups can be ionic salt groups, for example anionic salt groups, such as carboxylic acid and sulfonic acid salt groups, or cationic salt groups, such as amine salt groups and quaternary ammonium salt groups. The preferred hydrophilic groups are anionic groups and especially preferred are salts of carboxylic acid groups. A polymer is usually produced with carboxylic acid groups and then neutralised with a water soluble basic compound, such as an organic amine or an alkali metal hydroxide.

The concept “water-soluble” means in this connection that the resin-like materials, be made soluble in water, can be dispersed with a resin solids component of up to 25%, usually 1 to 20 percent by weight, without the aid of externally added tensids. The solution or dispersion frequently appears optically transparent or translucent, where the resin is present in the dispersed phase and has a mean particle size of 0.12 and less, usually less than 0.03 μm. The mean particle size of the water-soluble resin-like materials can be determined by a light scattering method.

The preferred lower molecular, water-soluble polymers are acrylic copolymers which have an anionic charge, preferably a carboxylic acid salt group and especially preferred a carboxylic acid group neutralised by an organic amine.

Among the lower molecular acrylic copolymers are polymers, produced by copolymerisation of an α, β ethylenic, unsaturated carboxylic acid with a methacrylic acid ester and/or acrylic acid ester, and in general acrylic polymers are suitable which comprise as the main component a methacrylate ester of a C₁-C₈ alcohol and a small proportion of an acrylate ester with a C₁-C₈ alcohol. The following compounds are common methacrylate esters and acrylate esters: ethyl acrylate, propylacrylate, isopropylacrylate, butylacrylate, isobutylacrylate, secondary butylacrylate, hexylacrylate, 2-ethylhexylacrylate, octylacrylate, methylmethacrylate, propylmethacrylate, isobutylmethacrylate, butylmethacrylate, secondary butylmethacrylate and tertiary butylmethacrylate.

The acryl polymers which are employed contain 0.1 to 20 percent by weight of a polymerised α, β ethylenic, unsaturated carboxylic acid unit. α, β ethylenic unsaturated carboxylic acid monomers which can be used are methyacrylic acid, acrylic acid, itaconic acid, ethacrylic acid, propylacrylic acid, isopropylacrylic acid and homologues of these acids. Methacrylic acid and acrylic acid are preferred. The percentage of acid is so set that the required acid number is created in the acrylic polymer. The acid number of the acrylic polymer should usually be so adjusted that it amounts to about 30 to 100 of the resin solids component. The numerical average molecular weights of the water-soluble acrylic polymers preferably lie in the range from 10 000 to 30 000.

The acrylic polymers can also contain hydroxyl side groups, which are obtained by copolymerisation of hydroxyalkylacrylates or hydroxyalkylmethylacrylates with the abovementioned acrylic esters. The hydroxyl groups-side groups provide places for subsequent curing, such as with an aminoplast or a masked isocyanate. 5 to 15 percent by weight of the utilised acrylic polymer are preferably from a hydroxyalkylacrylate or methacrylate ester. In general usable hydroxyalkylacrylates and methacrylates contain 1 to 8 carbon atoms in the alkyl group and are for example hydroxyethylacrylate, hydroxypropylacrylate, hydroxybutylacrylate, hydroxyethylmethacrylate, hydroxypropylmethacrylate, hydroxybutylmethacrylate, hydroxyhexylmethacrylate and hydroxyoctylmethacrylate for example.

Further vinyl copolymerisable compounds can be used, in order to form a part of the usable acrylic polymers, such as styrol, vinyltoluol, acrylamide, vinylxylol, allyl alcohol and acrylnitrile.

In an especially suitable acrylic polymer the polymer consist essentially of a hard component, namely either styrol or a lower alkylmethacrylate, wherein the acryl group contains 1 to 2 carbon atoms, or a mixture of styrol and lower alkylmethacrylate, such as ethylacrylate, a soft component, namely a lower alkylmethacrylate with 3 to 8 carbon atoms in the alkyl group or lower alkylacrylate with 2 to 8 carbon atoms in the alkyl group, a hydroxy lower alkylmethacrylate or acrylate with 1 to 4 carbon atoms in the alkyl group and an α, β-ethylenic, unsaturated carboxylic acid, as described above.

As well as water-soluble acrylic resins, polyesters which are produced from saturated or aromatic polycarboxylic acids and a polyol are suitable for the polymers having a lower molecular weight. Typical saturated aliphatic dicarboxylic acids are anhydrides with 2 to 10 carbon atoms, such as butandioic acid, azelainic acid and adipinic acid, which are suitable for the production of these polyesters. Examples of aromatic dibasic acids or their anhydrides are phthalic acid and trimellitic acid. The acid amount for the polyester is so set that the desired acid number is achieved, which should be 20 to 85 for the polyester. Many polyols can be converted with the above cited acids to produce the desired esters. Especially suitable diols are for example ethyleneglycol, 1, 4-butanediol, neopentylglycol, sorbitol, pentaerithritol and trimethylolpropane.

Alkyd resins, such as polymer esters, produced by condensation of polyhydric alcohol, such as glycerineethyleneglycol, and a drying fatty acid, such as linseed oil and tallol are also suitable as water-soluble polymers. A further component, which is usually added in order to achieve the desired acid number is for example α, β-ethylenic unsaturated dicarboxylic acid or the anhydride of the acid, such as maleic acid or maleic acid anhydride.

These alkyd resins should preferably have a numerical average molecular weight from 1 000 to 2 500 and an acid number from 20 to 85.

A further lower molecular carboxylic acid polymer which can be used for producing electro-deposition is a polymer of styrol and an ethylenic, unsaturated alcohol having 3 to 10 carbon atoms, such as allyl alcohol. The polymer can furthermore be converted with drying fatty acids and with an acid component such as those which have been recited above, in order to achieve the required acid number, usually in the range from 20 to 80. The numerical average molecular weights of the styrol-allyll-alcohol polymers usually lie in the range from 1 000 to 10 000.

Epoxide esters are also suitable as water-soluble resins of lower molecular weight. These materials are obtained by partial esterification of an epoxide resin with a customary drying fatty acid, such as those which have been recited above, and this resin is then esterified with an α, β-etheylenic, unsaturated dicarboxylic acid or an anhydride thereof, such as those which have been mentioned above. The epoxide resin itself is preferably a polyglycidylether of a bisphenol, such as bisphenol A.

Examples of further suitable resin-like polymers of lower molecular weight are the neutralisation products of unsaturated carboxylic acids, such as maleic acid or anhydride and a drying oil, such as linseed oil.

A further composition for the anodic electro-deposition comprises (A) 15 to 60 percent by weight water, (B) 15 to 60 percent by weight of one or more organic solvents, (C) 0.1 to 20 percent by weight of a copolymer of (a) 10 to 75 percent by weight of one or more copolymerisable α, β-olefinic, unsaturated compounds which are immiscible or partly miscible with water and (b) 25 to 90 percent by weight of one or more water-soluble copolymerisable N-vinyl compounds and (D) 10 to 79 percent by weight of one or more finely divided pigments or fillers or mixtures of pigments and fillers, dispersed in the mixture (A), (B), (C), wherein the sum of the percentages of (A), (B), (C) and (D) is 100. In particular component (C) in this is a copolymer which can be produced by solvent polymerisation for example. It is used in an amount of 0.1 to 20 percent by weight, preferably 3 to 8 percent by weight, and contains as copolymerisable units (a) 10 to 75 percent by weight, preferably 20 to 40 percent by weight, of one or more copolymerisable α, β-ethylenic, unsaturated compounds which are immiscible with water or only partly miscible, and (b) 25 to 90 percent by weight, preferably 60 to 80 percent by weight of one or more water-soluble copolymerisable N-vinyl compounds.

The sum of the percentages of (a) and (b) is 100. Preferred α, β-ethylenic, unsaturated compounds (a) which are immiscible with water or only partly miscible are vinyl esters of C₂-C₁₈-monocarboxylic acids, for example vinylacetate, vinylproprionate, vinylpivalate, vinyl-2-ethylhexanoate and vinylstearate and/or acrylic acid esters or methacrylic acid esters of C₄-C₁₈-alcohols, for example butylacrylate and butylmethacrylate, 2-ethylhexylacrylate, octylacrylate and octadecylacrylate. A particularly preferred monomer is vinylproprionate. Also suitable are derivatives of acrylamide or methacrylamide, vinylether and/or vinyl aromatics such as styrol, which are insoluble or only a little soluble in water.

Examples of preferred water-soluble N-vinyl compounds (b) are N-vinylpyrrolidone, N-vinylpiperidone and N-vinylimidazole.

Substances suitable for the cathodic electro-coating of metal articles will now be given. For example an emulsified mass is based on a copolymer, which includes tertiary amino groups and masked isocyanate groups and which is soluble or dispersible in water, in that it at least partially forms a salt with an acid, wherein the copolymer comprises as copolymerised units (A₁) 6 to 22 percent by weight of one or more monomers of tertiary aminomethacrylic acid or acrylic acid esters or acrylamides or methacrylamides with a tertiary amino group, (B₁) 21 to 40 percent by weight of a monomer adduct of an N-(1)-alkenylcyanate, wherein 1-alkenyl comprises 2 to 4 carbon atoms, and a CH-, OH- or NH-acidic masking means, (C₁) 0 to 35 percent by weight of one or more copolymerisable olefinic unsaturated compounds, which have active hydrogen atoms and which are reactive with isocyanate groups, and (D₁) 3 to 73 percent by weight of olefinic unsaturated compounds which are not recited under (A₁) to (C₁), selected from the group consisting of esters of acrylic acids and methacrylic acids with mono-alcohols with 1 to 18 carbon atoms, vinyl esters of carboxylic acids with 2 to 10 carbon atoms, vinyl aromatics, acryinitriles of unsaturated triglycerides, wherein the copolymer has a mean molecular weight of 1 000 to 20 000 and the percentages of (A₁) to (D₁) are 100 in total. Suitable components (A) are ethylenic unsaturated compounds which have tertiary amino groups, such as tertiary aminomethacrylic or acrylic acid esters, for example dialkylaminodalkylacrylate and methacrylate, wherein alkyl has 1 to 8 carbon atoms, for example N,N-dimethylaminoethylmethacrylate and N,N-diethylaminoethylacrylate or acrylamide or methacrylamide, which include a tertiary amino group, for example N,N-dimethylaminopropylacrylamide or methacrylamide and N,N-diethylaminopropylacrylamide or methacrylamide.

The binder contains 6 to 22, preferably 6 to 15 percent by weight of component (A₁) as a copolymerisable unit. The use of 6 to 10 percent by weight of the above recited acrylamides, which contain an amino group, is especially preferred.

Component (B₁) is an adduct of N-(1-alkenyl)isocyanate and a CH-, OH- or NH-acidic masking means. Suitable N-(1-alkenyl)isocyanates are those wherein alkenyl has 2 to 4 carbon atoms, preferably vinylisocyanate and/or propenylisocyanate. Examples of suitable masking means for the production of component (B₁) are monophenols, for example phenol, cresol and trimethylphenol, primary alcohols and secondary alcohols, for example isopropanol and cyclohexanol, tertiary alcohols, for example ter-butanol and ter-amyl alcohol, easily enolizable compounds, for example ethylacetoacetate, acetylacetone, malonic acid derivatives, for example malonic acid diesters with alcohols of 1 to 8 carbon atoms, malononitrile, secondary aromatic amines, for example M-methylaniline, N-methyltoluidine and N-phenyltoluidine, imides, for example succinimide and phthalimide, lactam, for example ε-caprolactam, δ-valerolactam and lauryllactam, as well as oximes, for example acetoneoxime, butanonoxime and cyclohexanonoxime. Especially preferred masking means for N-(1-alkenyl)isocyanate are ter-butanol, cyclohexanol and caprolactam.

The production of masked N-(alk-1-enyl)isocyanate, for example vinylisocyanate, can be effected by a process in the presence of a solvent for example. Approximately equimolar amounts are used for the conversion of N-(alk-1-enyl)isocyanate(phenolisocyanate) as a masking means. An excess of isocyanate should be avoided, since this can lead in the end to cross-linking. Component (B) is present in the copolymer in amounts from 20 to 40, preferably 25 to 33, percent by weight in the form of copolymerised units.

Suitable reactive monomers (C₁) for the copolymerisation are olefinic, unsaturated compounds which contain active hydrogen atoms, which are reactive to isocyanate groups, i.e. which carry OH- and NH-groups for example. Examples of these are monoesters of acrylic acids or acrylic acids with multivalent, especially divalent alcohols, for example hydroxyethylacrylate and methacrylate, ethoxypropylacrylate and methacrylate and hydroxybutylacrylate and methacrylate and monoesters of these acids with polyetherdioles, for example polypropyleneglycolacrylate and methacrylate, as well as allyl alcoholbut-1-ene-3,4-diol. N-methylolacrylamide and N-ethylolacrylamide can also be used. Esters of acrylic acids or methacrylic acids with dioles from 2 to 4 carbon atoms are especially preferred, especially hydroxypropylacrylate and hydroxyethylacrylate.

Component (C₁) is present in the copolymer in an amount from 0 to 35, preferably 20 to 30 percent by weight, as copolymerised units.

Suitable components (D₁) are copolymerisable, olefinic, unsaturated compounds which have not been recited under (A₁) to (C₁), for example esters of acrylic acids and methacrylic acids with monoalcohols with 1 to 18, preferably 1 to 8 carbon atoms, for example methylacrylate, ethylacrylate, butylacrylate, ethylhexylacrylate and methylmethacrylate. All further copolymiserable, unsaturated compounds can also be used, especially vinylesters of carboxylic acids with 2 to 10 carbon atoms, such as vinylacetate, vinyl aromatics, for example styrol, acrylnitrile and unsaturated triglycerides, e.g. isomerised linseed oil. The copolymers are preferably produced in polar solvents without OH groups, for example ethers, e.g. tetrahydrofuran, or esters, for example ethylacetate or N-butylacetate, in the presence of radical starters, such as azobiscarboxyamides, azobiscarboxylic acid nitriles and peroxides, in general at 60 to 120° C., preferably 60 to 90° C., in the present or absence of regulating means, for example ter-decylmercaptan and diisopropylxanthogendisulfide.

A further cationic coating substance comprises an aqueous medium and a resin-like binder dispersed therein, wherein the resin-like binder is produced by bringing into contact at least one starting material with a partially masked polyisocyanate compound at a temperature from 40° to 130° C. in a ratio by weight from 5 to 9 of the esters with 5 to 1 of the latter and subsequent neutralisation of the resultant product with an acid, wherein the starting material is at least one selected from the group consisting of

1. a mixture of a reaction product, produced by conversion of an epoxide resin with a basic amino compound with at least one basic amino group and a polyamide with at least one basic amino group in a ratio by weight from 1 to 9 of the reaction product to 9 to 1 of the polyamide, and

2. a further reaction product, produced by mixing the above recited reaction product with the polyamide at a temperature from 50° to 200° C., in a ratio by weight from 1 to 9 of the reaction product to 9 to 1 of the polyamide, wherein the partially masked polyisocyanate compound is present with at least one masked isocyanate group in the molecule and has on average more than 0 up to not more than 1 free isocyanate group per molecule.

The component 1. is a reaction product obtained by conversion of an epoxide resin with a basic amino compound. Usable epoxide resins are those which are obtained from a phenolic compound and epichlorhydrin, acidic epoxide resin, which has at least two epoxide groups per molecule, and usually have a molecular weight from about 200 to 4 000, preferably about 400 to 2 000. Particular examples of the resins of vinyl type are an epoxide resin produced from bisphenol A and epichlorhydrin, an epoxide resin produced from a hydrogenated bisphenol A and epichlorhydrin, an epoxide resin produced from bisphenol A and, β-methylenepichlorhydrin, polyglycidylether from novolak resin, etc., wherein an epoxide resin obtained from bisphenol A and epichlorhydrin is especially preferred. Such an epoxide resin of phenol type can be used together with a polyepoxy compound, such as polyglycidylether from ethyleneglycol, propyleneglycol, glycerine, trimethylolpropane and the like, multi-valent alcohols, polyglycidylesters of adipinic acid, phthalic acid, dimeric acid or the like, polycarboxylic acids, polyepoxides, obtainable by epoxiding alicyclic olefins and 1,2-polybutadiene, etc. The amount used can amount up to about 25 percent by weight.

Examples of basic amino acids which are converted with the epoxide resin are aliphatic or alicyclic amino compounds with a primary or secondary amino group. Preferred examples are monoamines, such as mono or dialkylamine, mono or dialalkanolamines and polyamines, such as polyalkylenepolyamine, etc. Suitable monoamines are mono or dialkylamines with say 1 to 18 carbon atoms, such as propylamine, butylamine, diethylamine, dipropylamine, etc. Examples of mono or dialkanolmonoamines are ethanolamine, propanolamine, diethanolamine, dipropanolamine, etc. Suitable examples of other monoamines are piperidine, cylcohexylamine, pyrrolidine, morpholine, etc. Examples of polyamines are ethylenediamine, hexamethylenediamine, triethylenetriamine, triethylenetetramine, tetraethylenepentamine, propylenediamine, dipropylenetriamine, butylenediamine, N⁻ aminoethanolamine, monoethylethylenediamine, diethyl-aminopropylamine, hydroxyethylaminopropylamine, monomethylaminopropylamine, piperazine, N⁻ methylpiperazine, N-aminoethylpiperazine, etc. Especially suitable are aliphatic mono or polyamines with a secondary amino group, such as diethylamine, diethanolamine, diethylenetriamine, monoethylethylenediamine, hydroxyethylaminopropylamine, etc., in respect of the reactivity with epoxide resin. An aromatic amine can be used in combination with an aliphatic or alicyclic amine in an amount such that the reaction product of epoxide resin and basic amine, neutralised with an acid, still remains dispersed in water. Examples of suitable aromatic amines are aniline, N-methylaniline, toluidine, benzylamine, m-xylylenediamine, m-phenylenediamine, 4,4-diaminodiphenylmethane, etc.

The conversion from epoxide resin with a basic amino group is effected in a manner known per se.

Examples of polyamide resin with a basic amino group, which is to be mixed in or converted with component (1), are those which include in the molecule at least one amino group or at least one amido group, which can react with the isocyanate group of the partially masked polyisocyanate compound. Examples are polyamides, produced by condensation of a dicarboxylic acid and polyamine, through conversion of a polyamine with an oligomer, produced by ring opening polymerisation of lactam, such as ε-caprolactam or polyesterpolyamide from alkanolamine and dicarboxylic acids, etc. are particularly preferred. The dicarboxylic acids are those given by the general formula

HOOC-R-COOH

wherein R is a saturated or unsaturated aliphatic hydrocarbon group or an aromatic hydrocarbon group with 1 to 34 carbon atoms. Preferred examples are phthalic acid, malonic acid, maleic acid, fumaric acid, butanedioic acid, azelainaic acid, adipinic acid, sebacic acid, dodecylbutanedioic acid, dimeric acid, etc. The polyamines are polyalkylenepolyamines with primary amino groups at both ends of the main chain, represented by the general formula

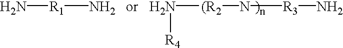

wherein R₁, R₂ and R₃ are aliphatic hydrocarbon groups with 2 to 6 carbon atoms, R₄ is hydrogen or an aliphatic hydrocarbon group with 1 to 3 carbon atoms and n is a whole number from 1 to 6. Preferred examples are ethylenediamine, propylenediamine, butylenediamine, hexamethylenediamine, tetraethylenepentamine, pentaethylenehexamine, hexamethyleneheptamine, hexaethyleneoctamine, diethylenetriamine, triethylenetetramine, bis(3-aminopropyl)amine, 1,3-bis(3-aminopropylamino)propane, etc. Suitable alkanol-amines include those with 2 to 6 carbon atoms, such as ethanolamine, propanolamine, hydroxyethylaminopropylamine, etc.

A suitable method for producing a electro-deposition film which can be deposited cathodically comprises the conversion of

(A₂) an unsaturated organic compound with a molecular weight from 300 to 30 000 with a carbon-carbon double bond, in an amount corresponding to an iodine number from 50 to 500, wherein the unsaturated organic compound is selected from the group consisting of (a) a polymer of a conjugated diolefin with 4 to 8 carbon atoms, (b) a copolymer of at least two conjugated diolefins containing 4 to 8 carbon atoms, (c) a copolymer of at least one conjugated diolefin containing 4 to 8 carbon atoms and a vinylmonomer with an ethylenic lack of saturation with 2 to 20 carbon atoms, (d) a natural oil, (e) a natural fat and (f) a mineral oil resin created by cationic polymerisation of mineral oil crack fractions with 4 to 10 carbon atoms with a Friedel-Crafts catalyst, wherein the unsaturated organic compound has epoxide groups bound thereon through carbon-carbon bonds has the formula

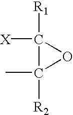

wherein R₁ and R₂ represent, independently of one another, hydrogen or methyl and X represents a hydrogen atom or a bond and when X represents a bond, the carbon atom to which R₁ is bound and the carbon atom to which R₂ is bound can form part of a main chain of the component (A),

wherein the amount of epoxy groups in the component (A₂) amounts to 0.05 to 0.2 mol per 100 g of the component (A₂),

with (B₂) a primary or secondary amino compound of the formula

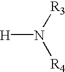

wherein R₃ and R₄ represent the same or different hydrocarbon groups with 1 to 10 carbon atoms, wherein each of R₃ and R₄ can be a hydrogen atom,

at a temperature from 100° to 200° C. to produce a resin-like substance which contains basic groups and hydroxide groups, through addition of a water soluble inorganic or organic acid to the resin-like substance, in order to make the resin-like substance water soluble, and mixing the resulting water soluble, resin-like substance with an aqueous or organic fluid medium, or performing the above conversion in the presence of a fluid medium.

Examples of such an unsaturated organic compound are natural oils and fats, such as linseed oil, tung oil, soyabean oil or dehydrogenated castor oil, and oils produced by heat treatment of natural oils and fats to increase their molecular weight. Examples of a fluid which contains unsaturated groups or of a solid polymer are polymers with a small degree of polymerisation of conjugated diolefins, usually with 4 to 8 carbon atoms, such as butadiene, isoprene or piperylene, copolymers with a small degree of polymerisation of two or more of these conjugated dienes or copolymers with a small degree of polymerisation of at least one of these conjugated olefins and a vinyl monomer with an ethylenic lack of saturation, usually with 2 to 20 carbon atoms, especially aliphatic or aromatic vinyl monomers, such as isobutylene, diisobutylene, acrylic or methacrylic acid or esters thereof, allyl alcohol or its esters, styrol, α-methylstyrol, vinyltoluol or divinylbenzol. These compounds can be used individually or in a mixture of two or more.

Moreover a suitable method is a method of electro-coating an electrically conductive surface, which serves as the cathode, comprising passing an electric current between the cathode and anode in contact with an aqueous electro-deposition composition,

wherein the electro-deposition composition comprises:

an acid-soluble, self-hardening, synthetic, organic resin with amino groups, hydroxyl groups and masked isocyanate groups, wherein the masked isocyanate groups are stable at room temperature in the presence of hydroxyl or amino groups and are reactive with hydroxyl groups or amino groups at raised temperatures and wherein the organic resin is derived from the reaction product of

1. an organic compound containing epoxy groups,

2. a primary or secondary amine and

3. a partially masked organic polyisocyanate.

The epoxide material which is used can be any monomeric or polymeric material with on average one or more epoxy groups per molecule. The monoepoxides can be used and the epoxide compound is preferably resin-like and is preferably a polyepoxide with two or more epoxy groups per molecule. The epoxy resin can in principle be any well-known epoxide. An especially useful class of polyepoxides are the polyglycidylethers of polyphenols, such as bisphenol A. These can for example be produced by etherification of a polyphenol with epichlorhydrin in the presence of an alkali. The phenol compound can for example be bis(4-hydroxyphenyl)-2,2-propane, 4,4′-dihydroxybenzophenone, bis(4-hydroxyphenyl)-1,1 -ethane, bis(4-hydroxyphenyl)-1,1-isobutane, bis(4-hydroxy-tertiary-butylphenyl)-2,2-propane, bis(2-hydroxynaphthyl)methane, 1,5-dihydroxynaphthylene, or the like. In many cases it is desirable to use polyepoxides with somewhat higher molecular weight, which contain aromatic groups.

Suitable, similar polyglycidylethers of multi-valent alcohols can be derived from multi-valent alcohols such as ethyleneglycol, diethyleneglycol, triethyleneglycol, 1,2-propyleneglycol, 1,4-propyleneglycol, 1,5-pentandiol, 1,2,6-hexantriol, glycerine, bis(4-hydroxycyclohexyl)2,2-propane and the like. Polyglydicylesters of polycarboxylic acids can also be used, which are produced for example by conversion of epichlorhydrin or similar epoxide compounds with an aliphatic or aromatic polycarboxylic acid, such as oxalic acid, butanedioic acid, glutaric acid, terephthalic acid, 2,6-napthylanedicarboxylic acid, dimerised linseed oil acid and the like.

Examples are glycidyladipate and glycidylphthalate. Also suitable are polyepoxides which are derived from epoxidation of olefinic unsaturated alicyclic compounds. Epoxides comprising in part one or more monoepoxides are included. These polyepoxides are not phenolic and are produced by epoxidation of alicyclic olefins, for example with oxygen and selected process catalysts, for example perbenzoic acid, produced via acetaldehydemonoperacetate or via peroxy acid. The epoxy-alicyclic ethers and esters are known among such polyepoxides. Further compounds containing epoxide and resins which contain diepoxides including nitrogen are described in U.S. Pat. No. 3,365,471, such as epoxide resins with 1,1-methylenebis(5-substituted)hydantoin (U.S. Pat. No. 3,391,097), diepoxides containing bisimide (U.S. Pat. No. 3,450,711), epoxydised aminemethyldiphenyloxide (U.S. Pat. No. 3,312,664), heterocyclic N,N′-diglycidyl compounds (U.S. Pat. No. 3,503,979), aminoepoxyphosphonates (GB-A 1 172 916), 1,3,5-triglycidylisocyanurate.

The partially or half capped or masked isocyanates, which can be used in the production of the compounds which are used, can be any polyisocyanates in which the proportion of the isocyanate groups has been so converted with a compound that the capped isocyanate part which is obtained is stable against hydroxyl or amine groups at room temperature but reacts with hydroxyl or amino groups at higher temperatures, usually above 90° C. and say 315° C. The semi-capped polyisocyanates should contain on average about one free reactive group.

In the production of partially masked organic polyisocyanates any suitable organic polyisocyanates can be used. Examples are aliphatic compounds, such as trimethylene-, tetramethylene-, pentamethylene-, hexamethylene-, 1,2-propylene-, 1,2-butylene-, 2,3-butylene-, 1,3-butylene-, ethylidene- and butylidenediisocyanates, the cycloalkylene compounds, such as 1,3-cyclopentane-, 1,4-cyclohexane- and 1,2-cyclohexanediisocyanates, the aromatic compounds, such as m-phenylene-, p-phenylene-, 4,4′-diphenyl-, 1,5-naphthaline- and 1,4-naphthalinediisocyanates, the aliphatic-aromatic compounds, such as 4,4′-diphenylenemethane-, 2,4- or 2,6-tolylene- or mixtures thereof, 4,4′-toluidine- und 1,4-xylylenediisocyanates, the aromatic compounds substituted in the ring, such as dianisidinediisocyanate, 4,4′-diphenyletherdiisocyanate- and chlordiphenylenediisocyanate, the -triisocyanates, such as triphenylmethane-4,4′,4″-triisocyanate, 1,3,5-triisocyanatebenzol and 2,4,6-triisocyanatetoluol and dietetraisocyanates, such as 4,4′-diphenyidimethylmethane-2,2′,5,5′-tetraisocyanate, the polymerised polyisocyanates, such as tolylenediisocyanatedimers und -trimers and the like.

The polyisocyanates which are employed should preferably have isocyanate groups with different reactivities, in order to facilitate the partial masking reaction.

Moreover organic polyisocyanates can represent a pre-polymer, derived from a polyalcohol, including polyetherpolyalcohol or polyesterpolyalcohol.

Some suitable aliphatic, cyclo-aliphatic, aromatic alkylmono alcohols and phenol compounds which can be used as masking means are for example lower aliphatic alcohols, such as methyl-, ethyl-, chlorethyl-, propyl-, butyl-, amyl-, hexyl-, heptyl-, octyl-, nonyl-, 3,3,5-trimethylhexanol-, decyl-and lauryl alcohols and the like and cyclo-aliphatic alcohols, such as for example cyclopentanol, cyclohexanol and the like; the aromatic alkylalcohols, such as phenylcarbinol, methylphenylcarbinol, ethyleneglycolmonoethylether, ethyleneglycolmonobutylether and the like; the phenol compounds, such as phenol itself, including phenols which are substituted. Examples are cresol, xylenol, nitrophenol, chlorphenol, ethylphenol, t-butylphenol and 2,5-di-t-butyl-4-hydroxytoluol.

Further masking means include tertiary hyroxylamines, such as diethylethanolamine and oximes, such as methylethylketoneoxime, acetoneoxime and cyclohexanoneoxime. The use of oximes and phenols is particularly desirable, since polyisocyantes which are masked with these means unmask at relatively low temperatures, without external addition of urathane-forming catalysts, such as tin catalysts.

Semi-capped organic polyisocyanates are produced by conversion of a sufficient amount of masking means with organic polyisocyanate, to provide a product with free isocyanate groups which remain.

The material containing epoxide is converted with an amine to produce an adduct. The amine which is employed can be any primary or secondary amine, preferably a secondary amine. The amine is preferably a water soluble amine compound. Examples of such amines include mono and dialkylamines, such as methylamine, ethylamine, propylamine, butylamine, dimethylamine, diethylamine, dipropylamine, dibutylamine, methylbutylamine and the like.

Examples of suitable polymers of lower molecular weight for cathodic lacquers which represent the acrylpolymers, which have been cited above and which in part comprise a monomer charge, are those which include an acyrlate or methacrylate containing a tertiary amine, such as dimethylaminoethylmethylacrylate, diethylaminoethylacrylate, diethylaminoacrylate and the like. These polymers can be dissolved and dispersed in water with the addition of acids, such as acetic acid which will disperse in water, or they can be quaternised with an alkylating means, such as methyliodide or dimethylsulfate, in order to get the required cationic charge. Moreover an acrylate or methacrylate containing a tertiary amino group, monomers such as methylvinylpyridine and the like can be used.

Further examples of polymer materials of lower molecular weight, which have an anionic charge, are a reaction product of polyepoxides, such a polyglycidylethers and of polyphenols, which have been recited above, converted with a secondary amine, such as dimethylaminodiethylamine. These adducts can then be neutralised with acids or be quaternised as described above, to provide the required cationic groups.

All the resins recited above can also contain resin-like materials which are insoluble in water. These materials are polymers and are in essence produced from hydrophobic, polymerisable reactants, such as ethylenic unsaturated monomer compositions, which each contain one or more polymerisable unsaturated ethylenic compounds which, when polymerised with one another, form polymers which are insoluble in water. The polymerisable, ethylenic, unsaturated compounds are represented by non-ionic monomers, such as the alkenyl-aromatic compounds, i.e. the styrol compounds, the derivatives of α,β-unsaturated monocarboxylic acids, such as acryl esters, acrylnitriles and methacrylic acid esters, derivatives of α,β-ethylenic unsaturated dicarboxylic acids, such as maleic acid esters, unsaturated alcohol esters, conjugated dienes, unsaturated ketones, unsaturated ethers and other polymerisable vinylidene compounds, such as vinyl chloride and vinylidenefluoride. Particular examples of such ethylenic unsaturated compounds are styrol, α-methylstyrol, α-ethylstyrol, dimethylstyrol, diethylstyrol, t-butylstyrol, vinylnaphthaline, hydroxystyrol, methoxystyrol, cyanostyrol, acetylstyrol, monochlorstyrol, dichlorstyrol and other halogen styrols, methylmethacrylate, ethylacrylate, butylacrylate, hexylacrylate, 2-ethylhexylacrylate, laurylmethacrylate, phenylacrylate, 2-hydroxybutylacrylate, 2-hydroxybutylmethacrylate, 4-hydroxybutylacrylate and 4-hydroxybutylmethacrylate, acrylnitrile, methacrylnitrile, acrylanilide, ethyl-α-chloracrylate, ethylmaleate, vinylacetate, vinylpropionate, vinylchloride, vinylbromide, vinylidenechloride, vinylidenefluoride, vinylmethylketone, methylisopropenylketone, vinylethylether, 1,3-butadiene and isoprene.

Preferred electro-deposition emulsions are inter alia anodic electro-deposition resin emulsions, especially from the company BASF, for example the deposition lacquer ZQ8-43225 from BASF Coatings, Münster.

As substance 4 with a detection function there can be used any materials, preferably in nano or micro particle sizes, which are endowed with a so-called detection function. The particle sizes amount to 10⁻⁵ to 10⁻⁸ cm, e.g. 10⁻⁶ to 10⁻⁷ cm, optionally 10⁻⁶ to 10⁻⁵ cm but even 10⁻⁷ to 10⁵ m, where the ranges can also comprise ten or a hundred times the range values.

By the concept “detection function” is understood the property of these substances whereby a physical, biological, biochemical or chemical characteristic specific thereto can be specifically recognised. The detection mechanisms can for example be effected on the basis of refraction of light, colour, fluorescence, phosphorescence, magnetism or radioactivity, but can also rely on chemical, especially electrochemical properties, such as amperometry, impedimetry or voltametry, redox processes, etc., biological and/or especially biochemical characteristics. Especially preferred substances with a detection function are latex beads, glass beads, graphite particles, coloured, fluorescing, phosphorescent, magnetic and/or radioactive particles and/or particles with chemical, biological and/or biochemical functions. Particles with a biological and/or biochemical function are especially enzymes, antibodies, antigens, haptens, DNA, RNA, oligo-nucleotides, peptides, oligopeptides, proteins, lectins, hormones, receptor antagonists or agonists, cells and microorganisms. The substance with a detection function is in particular an enzyme.

As an enzyme there can be used any enzymes which withstand the electro-deposition process or are compatible with the deposition lacquer emulsion employed. Especially preferred are oxidases, such as glucoseoxidase, peroxidase or lactateoxidase. However it is also conceivable to build up composite enzyme layers dependent on co-enzymes by multilayer formation, wherein NADH or NADPH-dependent dehydrogenases or PQQ-dependent dehydrogenases can be used in particular. Furthermore it is possible through multi-layer formation to combine also oxidases (e.g. ascorbateoxidase) and catalase, the latter as a protective layer. Other enzymes can moreover be functionally disposed in multiple layers.

In particular the method according to the invention can be used to carry out investigations in the field of chemical combinatorial analysis. For example different potentially catalytic materials can be specifically deposited by point-wise deposition on a flat electrode and the catalytic nature or even the activity of the deposited materials can be determined by specific sampling or sensing (e.g. electronically) thereof.

Among such materials are peptides, proteins and also inorganic substances, such as Cu/ZnO, Au/TiO₂ or transition metals doped with Na⁺. Catalysts based on classical catalysts such of those of the VIII transition group of the periodic system, especially Pt or Pd, are also conceivable.

Since the deposited polymers of the resin layer 3 have a backbone which allows the passage of molecules of a specific size, it is also possible through adjustment of the deposition parameters to exclude or allow the deposited layer to pass substances of different size, which are present together in a solution, in accordance with their size. A filter action is achieved. This can be used in multivarious ways—also for chemical combinatorial investigations. It can also be used in the depositions set out above with enzymes, especially in multilayer formations.

Water can optionally be added to the electro-deposition lacquer suspension available in the trade, where the water should be as pure as possible. Water of HPLC quality is preferred. Furthermore any other materials which are usually added to resin layers can be used. Such material are for example softeners, emulsifying agents, adhesion promoters, hydrophobing or hydrophilic materials, cross-linking agents, anti-foaming agents or the like.

Suitable additives which mainly exert the function of an emulsifier or cross-linking agent in the production and preparation of the electro-deposition lacquer suspension are anionic, cationic or amphophilic tensids, above all non-ionogen tensids. In this connection suitable commercially available products are set out below:

1. Triton (X-100, X-114, X-405 etc.): alkylphenylpolyethyleneglycol (Fluka)

2. Tween (20, 40, 60 etc.):

Tween 20: polyoxyethylenesorbitanemonolaurate (Merck)

Tween 40: polyoxyethylenesorbitanemonopalmitate (Merck)

Tween 60: polyoxyethylenesorbitanemonostearate (Merck)

Tween 65: polyoxyethylenesorbitanetristearate (Merck)

Tween 80: polyoxyethylenesorbitanemonooleate (Merck)

Tween 85: polyoxyethylenesorbitanetrioleate (Merck)

3. Nonidet P40: octyl-phenyl-polyethyleneglycol (Fluka)

4. Brij (35, 56, 58 etc.): (Merck)

Brij 35: polyoxyethylenelaurylether

Brij 56: polyoxyethylene-(10)-cetylether

Brij 58: polyoxyethylene-(20)-cetylether

5. octyl β-glucoside (Pierce)

6. octyl β-thioglucopyranoside (Pierce)

7. SDS: sodiumlauryl-sulfate (Fluka)

8. CHAPS: 3-[(3-cholamidopropyl)-dimethylammonio]-propanesulfonate (Fluka)

9. CHAPSO: 3-[(3-cholamidopropyl)-dimethylammonio]-2-hydroxy-propanesulfonate (Fluka)

In the method according to the invention an electro-deposition lacquer suspension is optionally thinned with water and is adjusted to a specific concentration of the substance 4. The ratio of emulsion to optionally further water to the substance 4 preferably amounts to 1-10:4-100:0.001-1, preferably 2-5:2-50:0.002-0.04. The electro-deposition can be effected in any vessel. The body (2), as a rule an electrode with a large area, is optionally pre-treated, dipped into the electrolyte solution and arranged opposite the electrode (5). A potential is applied between the two electrodes and is optionally controlled with a reference electrode, for example a calomel electrode or an Ag/AgCl standard electrode. The performance of the electro-deposition depends on the size of the electrode and the selected concentration of electro-deposition lacquer resin as well as on the tolerance of the enzyme for the process itself. Potential pulse profiles of 1500 mV for 1 s, then 700 mV for 1 s and then 0 mV for 5 s, in each case against the silver/silver chloride standard electrode with 2 cycles each are possible. Before use the electrode surface coated with enzyme/resin is kept overnight at 4 ΕC in 0.1 M phosphate buffer, pH 7, or another suitable buffer, in order to condition the electrode.

A calibration curve against known enzyme concentrations is taken from the electrode thus obtained. This curve is as a rule not linear over a wide range of concentration but linearity can be approximated over a short range of concentration. After determining the calibration curve the functionalised surface is ready for use, in order to measure unknown concentrations through the detection process for example.

A similar immobilisation can be undertaken for carbon electrodes which have been produced by means of thick film technology. The electrode is however not treated with a polishing paste before use. The applied potential profile can be for example 2000 mV for 1 s, then 700 mV for 1 s and then 0 mv for 5 s, in each case against an Ag/AgCl standard electrode with 2 cycles. The further procedures, such as recording the calibration curve and measurements can be effected as described above.

After producing an enzyme electrode for example this can in like manner be provided with a further layer (functionalised surface with protective layer). This second layer can contain a further enzyme or a catalytic substance or a mediator for electrons, hydronium ions or hydroxyl ions. For example a further layer can be applied with catalase on a GOD layer, which has been deposited as described above on a platinum electrode. The catalase protects against hydrogen peroxide which occurs for example. Thermal and aggressive reaction effects which are frequently noted with oxidase reactions are ameliorated, suppressed or avoided through the catalase layer.

The build up of multilayer sensors can be effected in a similar way as in the production of a protective layer, save that her more than two different layers are deposited in sequence. For example specific enzyme chains are conceivable, which detect partial sequences of biological enzymatic cycles.

As well as the large area deposition on a target electrode (2) targeted deposition can be effected by use of point-form electrode (5) (probe). Different enzymes according to changes of the deposition solutions are achieved on a surface through this. It is further possible to produce different enzyme concentrations on a surface, by point scanning of the surface of an electrode (2), so that a raster of points results, or by application of a potential field, whereby a continuum results (similar to the Hull cell). It is possible with the aid of this arrangement to undertake chemical combinatorial investigations. The process can also be inverted. Deposited detection functionalised resin surface can be dissolved specifically with a probe, so that a point raster or a density continuum results.

The articles according to the invention can preferably be used in the field of bio-sensor techniques of biological and chemical combinatorial analysis, chemical diagnostics, for example screening and catalyst research. Sensors are provided through the invention with multiple functions in a simple way. It is possible to allow the most varies operations to proceed simultaneously on one sensor surface.

### EXAMPLES

Reagents employed:

Acros, New Jersey, USA

N-Hydroxy-succinimide 98%

J. T. Baker, Deventer, Holland

D(+) Glucose-1-hydrate z.A.

Water for the HPLC

Hydrogen peroxide 30% z.A.

BASF, Münster, Germany

Deposition lacquer ZQ8-43225

Janssen Chimica, Geel, Belgium

Lithiumperchlorate z.A.

Merck, Darmstadt, Germany

Hexachloroplatinic-(IV)-acid-hexahydrate for Synthesis

Potassium chloride Suprapur

Sigma, Deisenhofen, Germany

EDAC 1-Ethyl-3-(3-dimethylamino-propyl)carbodiimide

Glucoseoxidase type X-S from Aspergillus Niger 100000-250000 U/g

catalase from bovine liver 2100 U/mg

Latex Beads 0,431 μm Carboxylate modified and fluorescent

Strem Chemicals, Newburyport, USA

Hexaamminruthenium(III)chloride

### Example 1

1) Glucose Sensor:

250 μl EPD lacquer suspension were thinned with 5 ml water (for HPLC). An enzyme concentration of 3 mg glucoseoxidase/ml solution was set. The following potential pulse profile was applied to a platinum electrode (p 1 mm) in this solution (1500 mV for 1 s, then 700 mV for 1 s and then 0 mV for 5 s in each case/AgCl. 2 cycles were performed). The electrode was treated before use with 3 μm, 1 μm and 0.3 μm polishing paste and then platinised. Before recording the calibration curve it was kept overnight at 4° C. in 0.1 M phosphate buffer, pH 7.

The recording of the calibration curve (FIG. 2) was effected in 20 ml phosphate buffer, pH 7, by addition of various volumes of a 100 mM glucose solution. The potential applied to the coated electrode amounted to 600 mV/AgCl. A similar immobilisation method was used for the carbon electrode produced by means of thick film technology, but with the difference that the electrode was not treated before use with polishing pastes. The applied potential profile ran as follows, 2000 mV for 1 s, then 700 mV for 1 s and then 0 mV for 5 s in each case/AgCl. 2 cycles were performed. The recording of the calibration curve (FIG. 3) took place according to the method described above.

### Example 2

Glucose Sensor with Catalase Protective Layer

The procedure was as in Example 1. Instead of the GOD enzyme concentration of 3 mg/ml solution 3 mg catalase per ml solution were used. The 1 mm φ platinum disc electrodes employed were pretreated as in Example 1. The applied potential pulse profile ran 2000 mV for 1 s, then 800 mV for 1 s and then 0 mV for 5 s, in each case/AgCl. An electrode treated in the same way served as the comparison electrode, being modified with GOD film (FIG. 4) and being an unmodified platinum electrode (FIG. 5).

The measurement of the hydrogen peroxide sensitivity took place in 20 ml 100 mM phosphate buffer, pH 7, through addition of different volumes of a 2 mM hydrogen peroxide solution. The potential applied to the coated electrode amounted to 600 mV/AgCl.

### Example 3

Building Up Multilayer Sensors (Sequential Deposition):

The procedure was as in Example 1, wherein a thin layer was formed. The deposition solution was then changed and the deposition operation repeated.

### Example 4

Deposition of Sensor Arrays:

The procedure was as in Example 1 with the exception of the polishing step. A homogenous polymer layer laterally bounded in accordance with the dimension of the conductive parts of the structure resulted. (Potential pulse profile: 2000 mV for 0.5 s//700 mV for 1 s//0 mV for 5 s//one cycle). The deposition was carried out on a micro-strip structure (1000 μm*20 μm) in a solution containing GOD, cf. also FIG. 11.

### Example 5

Inclusion of Fluorescent Beads

The procedure was as in Example 4. A polymer film resulted which was restricted to the dimensions of the coated micro-structure. The polymer solution contained 250 μl EPD lacquer in 5 ml water, to which was added about 10 μl of the bead suspension. (Potential pulse profile: 2000 mV for 0.5 s//800 mV for 1 s//0 mV for 5 s//one cycle). The coated micro-structure involved a three electrode system. The first figure (FIG. 6) shows the micro-structure with two coated micro-strip electrodes under normal light. The L-shaped strip electrode was coated with a GOD film (the same potential pulse profile as for the bead deposition), while the smaller, straight strip electrode was provided with the bead film. The middle micro-strip electrode is an Ag/AgCl reference electrode.

By comparison with the above Figure only the film with added fluorescent latex beads which has been deposited selectively on the strip electrode can be detected on the exposure (FIG. 6) of the same microstructure made by means of a fluorescent microscope.

The latex beads served in this case as a test substance, in order to test the possibility of the inclusion of larger and smaller particles in the film. The inclusion of particles (beads) modified with detection elements or anchor groups is conceivable, in order to produce so-called “screening assays” in a simple way. These can then be evaluated by means of various detection methods, such as fluorescence for example. A further use presenting itself would be the construction of combinatorial libraries.

### Example 6

Micro-Structured (Local) Deposition on Conducting Surfaces (Non-Manual Addressability):

The micro-structured deposition of polymers on conductive surfaces was effected by means of electrochemical raster probe microscopy. In this method a (nano) micro-electrode is approached to the surface (5-10 μm) by means of positive or negative feedback based on a change of the Faraday current at the micro-electrode in an electrolyte solution containing a redox species (5 mM ruthenium hexaammin solution) [C. Krantz, M. Ludwig, H. E. Gaub, W. Schumann, Adv. Materials, 7 (1995) 568-571]. The electrolyte solution is then exchanged with the polymer solution (GOD polymer solution with 0.5 mg KCl), the conducting surface switched to the working electrode and the approached micro-electrode to the counter-electrode. As well as glass sheathed platinum disc electrodes carbon fibre micro-electrodes were also used as micro-electrodes for the investigations (carbon fibres φ 8 μm, Sigrafil insulated with EDP lacquer (Electrodeposition Paint). The conducting surfaces consisted of a disc gold electrode (φ 3 mm).

The pH value can now be altered locally in the volume between the working and counter-electrodes by altering the potential. This then leads to a local precipitation of the polymer below the micro-electrode. The selected potential pulse profile runs initially 800 mV for 1 s and then 0 mV of 10 s, in each case/AgCl. This pulse profile was repeated 5 times. By altering the starting pulse and the number of cycles films with different densities and sizes were produced (FIG. 7). The resultant polymer points on the gold surface had a diameter of 50 to 100 μm.

### Example 7

Local Dissolution of the Polymer Film (Non-Manual Dissolution)

In the local dissolution of the polymer film the film is selectively dissolved by a locally generated change in the pH value at this place. The dissolved polymer can then be removed by a rinsing operation. The places on the surface now accessible again can now be functionalised again, cf. FIG. 12.

For the local dissolution of the film a gold disc electrode (φ 3 mm) was initially fully coated with the polymer film. A glass sheathed platinum disc electrode (φ 50 μm) was then approached to the film surface (as in Example 6). A potential is then applied to the micro-electrode which leads to cathodic decomposition of water and thus to creation of OH⁻ below the micro-electrode. The film can be locally dissolved by this change in pH value.

A potential pulse of −2000 mV/AgCl was applied for 1 s each time and then repeated several times. The L seen in the picture (FIG. 8) is a partially dissolved film at this place which arose through a lateral displacement of the electrode during the pulse operation. For comparison therewith a scratch has been made on the film surface.

### Example 8

Modification of the Polymer Films

Since the EDPs have functional groups, such as acid or amino groups, there are good possibilities for their covalent modification. The modification can be effected both before the deposition and after the deposition, depending on the requirements. Also the nature of the modification means to be used is only dependent on its stability in the polymer solution. The film is modified with redox active groups for example. A suspension consisting of 150 μl lacquer and 3 ml phosphate buffer (0.1 M and pH 7), 0.5 mM EDAC and 20 mM N-hydroxy succinimide was added for this. This is then well shaken for 3 h at room temperature and then deposited by means of the electro-deposition.

Potential pulse profile: 2000 mV for 0.2 s//700 mV for 1 s//0 mV for 5 s//5 cycles. The electrode thus modified was immersed for 24 hours in a 10 mM [Os(bpy)₂histamine)Cl]Cl solution at room temperature. The electrode was then thoroughly rinsed with water and kept for several hours in water. After this step several CV's (FIG. 9) and DPV's (FIG. 10) were taken from this film in 0.1 M lithium perchlorate solution. As can be seen from the two pictures redox active components (the osmium complex) are present on the electrode.

1. Comprising an optionally electrically conductive body (2) and a resin layer (3) containing at least one substance (4) with a detection function applied thereto, a method of producing a surface (1) functionalised with detection elements, characterized in that
a) a resin layer (3) is deposited on the body (2) by electro-deposition,
b) the resin layer (3) comprises resins derived from polymerisable, ion-carrying monomers which are deposited by changing the pH value and
c) the substance(s) and optionally further additives is/are deposited with the resin from a resin emulsion on the body (2) during the electro-deposition.

2. A method according to claim 1, wherein the resin of the resin layer (3) is derived from acrylic monomers.

3. A method according to claim 1 or 2, wherein the electro-deposition is effected by creation of H⁺ ions on the body (2).

4. A method according to claim 1 or 2, wherein the electro-deposition is effected by creation of OH⁻ ions on the body (2).

5. A method according to any of claims 1 to 3, wherein the electrolyte used for the electro-deposition comprises an emulsion of water and a resin preparation on the basis of a copolymerisate which contains carboxyl groups, optionally also masked isocyanate groups, hydroxyl and/or ether groups and which is soluble or dispersible in water through at least partial salt formation with ammonia or an organic or inorganic base.

6. A method according to any of claims 1, 2 and 4, wherein the electrolyte used for the electro-deposition comprises an emulsion of water and a resin preparation on the basis of a monomer, oligomer or polymer having organic amino groups, wherein the resin preparation is of basic nature and the amino groups are optionally substituted with C₁-C₆ alkyl groups, which for their part are optionally substituted with OH or optionally substituted amino groups, and/or are interrupted by heteroatoms, selected from S, O, NR₁, wherein R₁ signifies a C₁-C₆ alkyl group.

7. A method according to any of claims 1 to 6, wherein as an optionally electrically conductive body (2) there is used a noble metal selected from gold, silver, platinum, palladium, iridium, rhenium, mercury, ruthenium and osmium, preferably platinum.

8. A method according to any of claims 1 to 7, wherein at least two electro-deposits with different substances (4) are deposited with on the body (2).

9. A method according to any of claims 1 to 8, wherein the deposition is effected in an array.

10. A method according to any of claims 1 to 9, wherein the deposition is effected by means of raster probe microscopy, wherein resin points with detection elements optionally of different densities and sizes are created.

11. A method of producing a surface functionalised with detection elements, wherein a deposition according to any of claims 1-10 is initially effected and then this is selectively cathodically or anodically dissolved to form resin areas with detection elements, optionally of different densities and sizes.

12. A method according to any of claims 1 to 11, wherein a substance (4) with a detection function is selected from latex beads, glass beads, graphite particles, colouring, fluorescing, phosphorescing, magnetic and/or radioactive particles and/or particles with chemical, especially electrochemical properties, such as amperometry, impedimetry, voltametry, redox processes, etc., a biological and/or biochemical function.

13. A method according to claim 12, wherein substance (4) signifies particles with a biological and/or biochemical function.

14. A method according to claim 13, wherein a substance (4) is selected from at least one of enzymes, antibodies, antigens, haptens, DNA, RNA, oligonucleotides, peptides, oligopeptides, proteins, lectins, hormones, receptor antagonists or agonists, cells and microorganisms.

15. A method according to claim 13 or 14, wherein substance (4) is an enzyme.

16. A method according to claim 12, wherein the substance (4) is an inorganic catalyst, selected from Cu/ZnO, Au/TiO₂, transition metals doped with Na⁺ and/or metals of the VIII transition group.

17. A method according to any of claims 1 to 16, wherein deposition of different thickness and/or density is achieved by altering the process parameters, especially the level of the electric potential, the diffusion, the condensation and/or the layer thickness of the electrolytes.

18. An article obtainable according to any of claims 1 to 17.

19. An article according to claim 18, wherein the body (2) consists of platinum and the substance (4) is GOD.

20. An article according to claim 18, produced according to claim 6, wherein a first layer on the body (2) contains GOD and a second layer thereon contains catalase.
